(19) United States
(12) Patent Application Publication
(10) Pub. No.: US 2022/0415454 A1
(43) Pub. Date: Dec. 29, 2022

(54) Systems and Methods for Estimating Treatment Effects in Randomized Trials Using Covariate Adjusted Stratification and Pseudovalue Regression

(71) Applicant: Unlearn.AI, Inc., San Francisco, CA (US)

(72) Inventors: Alejandro Schuler da Costa Ferro, San Francisco, CA (US); David Putnam Miller, Albany, CA (US); Yunfan Li, San Francisco, CA (US); Alyssa Vanderbeek, San Francisco, CA (US)

(73) Assignee: Unlearn.AI, Inc., San Francisco, CA (US)

(21) Appl. No.: 17/808,954

(22) Filed: Jun. 24, 2022

Related U.S. Application Data
(60) Provisional application No. 63/363,796, filed on Apr. 28, 2022.
(60) Provisional application No. 63/214,643, filed on Jun. 24, 2021.

(51) Int. Cl. G16H 10/20; G16H 50/20

(52) U.S. Cl. CPC G16H 10/20; G16H 50/20

(57) ABSTRACT

Systems and methods for estimating treatment effects in randomized controlled trials using covariate adjusted stratification and pseudovalue regression in accordance with embodiments of the invention are illustrated. One embodiment includes a method for estimating treatment effects in randomized controlled trials, where the method includes receiving external data of previous randomized clinical trials. The method further includes generating sets of one or more subject characteristics of a plurality of trial subjects, estimating binary outcomes of trial subjects using a stratification process, and estimating time-to-event (TTE) treatment effects of trial subjects using pseudovalue regression.

### CROSS-REFERENCE TO RELATED APPLICATIONS

The current application claims the benefit of and priority under 35 U.S.C. § 119(e) to U.S. Provisional Patent Application No. 63/214,643 entitled “Systems and Methods for Randomized Trials via Prognostic Score Stratification” filed Jun. 24, 2021, and U.S. Provisional Patent Application No. 63/363,796 entitled “RMST Pseudovalue Regression Variance” filed Apr. 28, 2022, the disclosures of which are hereby incorporated by reference in their entireties for all purposes.

### FIELD OF THE INVENTION

The present invention generally relates to clinical trial design and, more specifically, improving statistical power to detect treatment effects using covariates derived from generative models for stratification and/or pseudovalue regression.

### BACKGROUND

Clinical research and clinical trials aim to study the safety and efficacy of biomedical or behavioral interventions on humans. When new drugs and medical devices are invented, they must undergo rigorous trials to generate data on its efficacy and safety in order to be approved by the relevant authorities for clinical use. Test articles that do not produce satisfactory safety or efficacy levels will not be approved for mass commercial use.

Randomized controlled trials (RCT) are one method used to conduct a clinical trial. An RCT generally has two arms, namely the treatment arm and the control arm. Enrolled subjects are assigned to each arm randomly, and the efficacy of a proposed new treatment is determined by comparing trial outcomes of subjects enrolled in the treatment arm that received the new treatment against trial outcomes of subjects enrolled in the control arm that received an existing treatment. While outcomes are influenced by participants' individual characteristics due to the subtle ways in which they differ from each other, RCTs allows statisticians to have control over these influences. A well-designed RCT may provide reliable indication on not only the trial outcome, but also information on possible adverse effects of the experiment.

Covariate adjustment refers to the controlling of baseline characteristics of trial subjects when estimating treatment effects. In most cases, trial outcomes are correlated to the baseline characteristics of the trial subjects. In the context of an RCT, covariate adjustment is an effective tool to assist with estimating treatment effects. Since baseline characteristics are collected and measured before random assignments, statistician retain the ability to test for treatment effects across the randomized trial groups by adjusting known covariates of the randomized trial groups.

### SUMMARY OF THE INVENTION

Systems and methods for estimating treatment effects in randomized controlled trials using covariate adjusted stratification and pseudovalue regression in accordance with embodiments of the invention are illustrated. One embodiment includes a method for estimating treatment effects in randomized controlled trials, where the method includes receiving external data of previous randomized clinical trials. The method further includes generating sets of one or more subject characteristics of a plurality of trial subjects, estimating binary outcomes of trial subjects using a stratification process, and estimating time-to-event (TTE) treatment effects of trial subjects using pseudovalue regression.

In another embodiment, the method includes steps for estimating binary outcomes of trial subjects using a stratification process, where the method includes training a prognostic model using the received external data, generating outcome predictions for trial subjects using the prognostic model, defining a variable to stratify the trial subjects based on the outcome predictions, stratifying all trial subjects by the variable in to a plurality of strata, and estimating treatment outcomes for trial subjects in all strata.

In a further embodiment, the method further includes steps for estimating TTE treatment effects of trial subjects using pseudovalue regression, where the method includes training a prognostic model using the received external data, generating prognostic scores of trial subjects using the prognostic model and the generated trial subjects' subject characteristics, and estimating TTE treatment effects for trial subjects using a pseudovalue regression model and the prognostic scores.

In still another embodiment, the sets of one or more characteristics of a plurality of trial subjects include baseline covariates of trial subjects, and treatment assignments of trial subjects.

In a still further embodiment, the prognostic model is a generative model.

In yet another embodiment, the prognostic model is a generalized linear model.

In a yet further embodiment, the prognostic model is a simple rules-based model.

In another additional embodiment, the prognostic model is a model-based generative machine learning model.

In a further additional embodiment again, estimating TTE treatment effects includes estimating restricted mean survival times of trial subjects.

In another embodiment again, the method further includes designing clinical studies based on estimated treatment effects.

One embodiment includes a non-transitory machine readable medium containing processor instructions for estimating treatment effects in randomized controlled trials using covariate adjusted stratification and pseudovalue regression, where execution of the instructions by a processor causes the processor to perform a process that includes receiving external data of previous randomized clinical trials. The method further includes generating sets of one or more subject characteristics of a plurality of trial subjects, estimating binary outcomes of trial subjects using a stratification process, and estimating time-to-event (TTE) treatment effects of trial subjects using pseudovalue regression.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 is a flowchart of a process to estimate treatment effects in a randomized controlled trial.

FIG. 2 is a flow chart of a process to incorporate strata based upon a generative model in the design of a randomized controlled trial in accordance with an embodiment of the invention.

FIG. 3 is a flow chart of a process to estimate treatment effects for TTE outcomes in accordance with an embodiment of the invention.

FIG. 4 is a diagram of a network where a process that estimates treatment effects may be implemented on in accordance with an embodiment of the invention

FIG. 5 is a high-level block diagram of a system for a process estimating treatment effects to be implemented on in accordance with an embodiment of the invention.

FIG. 6 is a high-level block diagram of an application that executes a process estimating treatment effects in accordance with an embodiment of the invention.

### DETAILED DESCRIPTION

Systems and methods in accordance with some embodiments of the invention can estimate treatment effects in randomized controlled trials (RCTs). In several embodiments, the treatment effect may be estimated from the outcomes under control and treatment conditions for subjects enrolled in the trial. Systems and methods in accordance with various embodiments of the invention can estimate treatment outcomes using covariate adjusted stratification. In many embodiments, the treatment effect for an event outcome may be evaluated based on differences in the time to the event under control and treatment conditions. Systems and methods in accordance with many embodiments of the invention can estimate time to treatment effect using covariate adjusted pseudovalue regression.

Processes in accordance with certain embodiments of the invention can improve RCT design by reducing the sample size required for the trial. In many embodiments, processes can reduce the variance of estimations performed, which can improve the accuracy of the estimations.

RCTs often require sufficiently large sample sizes for results to be representative. However, large sample sizes of trial subjects can also increase the difficulty of enrolling an adequate number of participants, which can make it challenging to complete the study or provide sufficient power to estimate treatment effects. Embodiments of the invention can solve this problem through data stratification. In many embodiments, trial subjects may be partitioned into nonoverlapping groups by a certain characteristic of the trial subjects. In several embodiments, stratification of trial subjects may be performed multiple times based on multiple subject characteristics. Machine learning models in accordance with a number of embodiments of the invention can be used to estimate outcomes under control conditions, which can be used to identify optimal groupings that may be used to stratify the trial subjects.

In RCTs, time-to-event (TTE) analyses are important for their ability to establish a time frame by which a major clinical event may occur in the trial. However, in clinical research and trials, there will always be subjects dropping out from the trial before the clinical event of interest is ever reached. A well-conducted RCT will typically have approximately 10 to 20 percent of trial subjects leaving the study before the intended time of follow-up. The lost subjects are treated as censored data for the purposes of the trial as of the last known follow-up. Cumulative amounts of censored data can affect the established time frame to major clinical events in the trial, which consequently affects the estimation of treatment effects. Embodiments of the invention can solve this problem by using pseudovalue regression to analyze TTE treatment effects of trial subjects. In certain embodiments, pseudovalue regression is applied censored data to estimate TTE treatment effects.

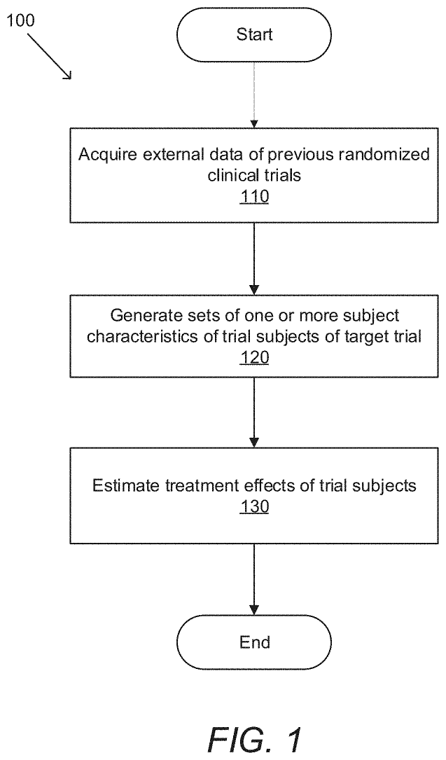

An example process of estimating treatment effects in RCTs in accordance with many embodiments of the invention is illustrated in FIG. 1. In many embodiments, process 100 acquires (110) external data of trial subjects from previous randomized clinical trials. In some embodiments, external data may be from high quality observational studies. External data in accordance with several embodiments of the invention may include subject characteristics of trial subjects, and/or their eventual trial outcomes from the previous randomized clinical trials. In many embodiments, prognostic models are trained with acquired external data, and the models can be used to estimate outcomes for patients under control conditions. Embodiments of the invention can leverage these estimated outcomes to improve precision of estimated treatment effects, which will be explained in further detail below.

Process 100 generates (120) sets of one or more subject characteristics of trial subjects of a target trial. In certain embodiments, subject characteristics include baseline covariates of each trial subject and subjects' treatment arm assignments. Subject characteristics may be used individually, or in combinations of two or more in the estimation of treatment effects discussed in detail below.

Process 100 estimates (130) treatment effects of trial subjects. In many embodiments, estimated treatment effects include treatment outcomes, and TTE treatment effects. In several embodiments, treatment outcomes may be binary in that they account for whether trial subjects have achieved the desired treatment outcome or not. Binary treatment outcomes may be estimated using a stratified analysis whereby the entirety of trial subjects is partitioned into nonoverlapping groups known as strata by a certain subject characteristic that all trial subjects possess, thus allowing researchers to observe the correlation between certain subject characteristics and the binary trial outcome. In many embodiments, treatment assignments may be independent of the subjects' strata, as trial subjects are randomly assigned to either the control arm or the treatment arm of the trial before stratification takes place.

Time-to-event (TTE) analyses establish a time frame by which a major clinical event may occur in the trial, and can be another indicator of the efficacy of the new treatment on trial. The event of interest in many embodiments may be whether the trial subject obtains the desired treatment outcome. In a number of embodiments, treatment effects can include TTE treatment effects. In accordance with embodiments of the invention, TTE treatment effects can allow researchers to observe how TTE for certain events vary among the trial subjects. However, TTE treatment effects may be affected by trial subjects dropping out of the trial before obtaining the events of interest. Therefore, in many embodiments, TTE treatment effects for trial subjects including censored subjects may be estimated to maintain an accurate reflection of trial results based on the original trial enrollment. In several embodiments, TTE treatment effects are estimated using parametric regression models including the pseudovalue regression method, which will be discussed further in detail below.

In numerous embodiments, clinical studies may be designed based on estimated treatment effects. In many embodiments, clinical studies designed based on estimated treatment effects can maintain a desired level of study power while keeping sample sizes small to save costs. Variances of the studies may also be reduced to achieve maximum accuracy possible in accordance with embodiments of the invention.

While specific processes for estimating treatment effects in RCTs are described above, any of a variety of processes can be utilized to estimate treatment effects in RCTs as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

### Estimating Treatment Effects for Binary Outcomes

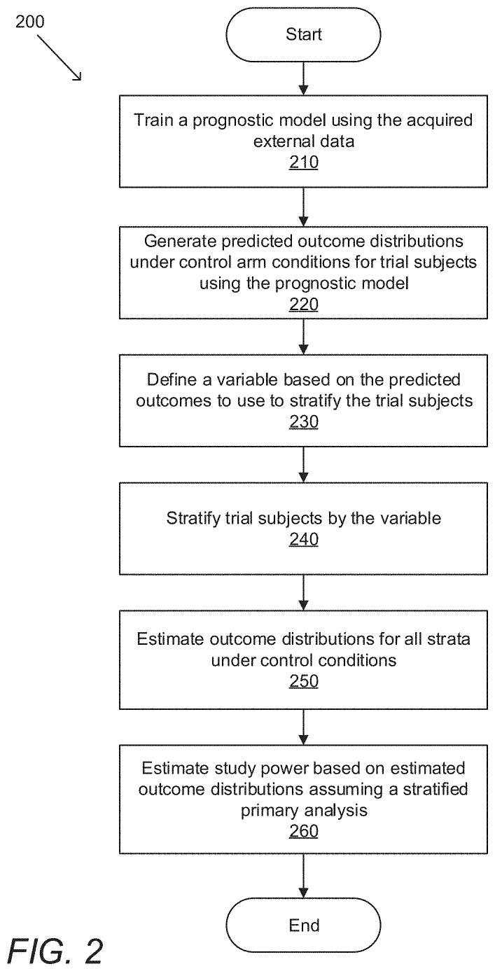

Estimating treatment effects for binary outcomes using stratification is a multi-step process. A conceptual illustration of the stratification and estimation process is illustrated in FIG. 2. Process 200 trains (210) a prognostic model using acquired external data from previous trials. In some embodiments, external data may be from high quality observational studies. External data in accordance with several embodiments of the invention may include subject characteristics of trial subjects, and/or their eventual trial outcomes from the previous randomized clinical trials. In certain embodiments, the prognostic model may be a generative model. In a number of embodiments, the prognostic model may have binary, categorical, continuous and time-to-event outputs that are subsequently used to derive the probability of a binary outcome for each trial participant.

Process 200 generates (220) predicted outcomes under control arm conditions for trial subjects using the trained prognostic model. In several embodiments, prognostic models generate outcome predictions using the entire set of one or more subject characteristics. As the outcome of interest is often binary in RCTs, outcome predictions generated in many embodiments of the invention may also be binary in nature as the scores predict the outcome probability between the two possible outcomes. If binary outcomes are defined by some underlying continuous variable, predictions of the continuous variable itself may be used as stratifying variables in certain embodiments of the invention. In several embodiments, selection of the stratifying variable may be determined jointly by the definition of the outcome and the expected variance and sample size reduction possible.

In many embodiments, the stratification processes use the framework of a traditional Cochran-Mantel-Haenszel (CMH) test. The CMH method uses a stratifying variable to separate the trial subjects into a series of 2×2 contingency tables illustrated as follows:

TABLE 1
2 × 2 table for a binary outcome of trial
subjects in both treatment and control arms

| | Has outcome | Does not have outcome |
|---|---|---|
| Treatment arm | A | B |
| Control arm | C | D |

When all trial outcomes are observed, cell A would represent the number of subjects assigned to the treatment arm that obtained the desired outcome. Cell B represents the number of subjects assigned to the treatment arm that did not obtain the desired outcome. The same interpretation follows for C and D on the control arm.

Process 200 defines (230) a variable X based on the predicted outcomes to use to stratify the trial subjects. In several embodiments, X may be defined as the probability pⱼ of observing outcome Y and can be ordinal. In certain embodiments, process 200 can define the variable X by combining all treatment outcome predictions aᵢ and separating all aᵢ into a number of strata denoted by j. In the context of a trial that uses treatment outcome predictions in conjunction with the CMH method, processes in accordance with certain embodiments of the invention can separate the trial subjects into strata based on their probability of a binary outcome occurring during the study. In several embodiments, this can allow for a more flexible application of the prognostic information in a range of baseline variables to create strata, where said strata are based on outcome predictions under control conditions. For a trial that is not stratified with outcome predictions under the CMH method, the stratifying methodology of the trial could be replaced by strata defined by treatment outcome predictions since strata defined by treatment outcome predictions incorporates the entire set of one or more subject characteristics.

In several embodiments, process 200 may define (230) stratifying variables using GLMs and perform the proposed covariate adjusted analysis. GLMs can allow for multiple additional covariates, in addition to the proposed stratification variable, to be included in the model stratification analysis. Let Y={0,1} be the outcome vector that denotes outcomes for subjects i, and ZX, be the vector of covariates for subjects i. In many embodiments, GLM may be defined as g(X)=X′β. According to a number of embodiments of the invention, g may be a link function including but not limited to logit, Poisson, and log-binomial functions.

Process 200 stratifies (240) the trial subjects by the variable X. into j strata, where j=1,2, . . . , J. In many embodiments, p₀ⱼ and p₁ⱼ denote the expected outcome probabilities under control and treatment arms respectively for a stratum xⱼ, and n₀ⱼ and n₁ⱼ represent the observed counts of subjects in control and treatment arms respectively for each stratum. Process 200 estimates (250) outcomes distributions for all strata under control conditions. In several embodiments, process 200 tests the null hypothesis H₀: ψ̅=ψ₀ against an alternative H₁: ψ̅≠ψ₀, where ψ̅ is the estimate of marginal treatment effects. Sampling distributions of ψ under the null and alternative hypotheses may be given by N(ψ₀, \hat{V}₀) and N(\hat{ψ}, \hat{V}₁) respectively according to many embodiments of the invention, where V denotes the variances of the estimates of marginal treatment effects. In certain embodiments, processes can estimate marginal treatment effects and variances of the estimates based on the number of strata, and the treatment outcome predictions for each stratum. Estimated marginal treatment effects and variances under the alternative hypothesis may be both a weighted sum of J strata-level values, where weights wⱼ may be defined by the observed counts n₀ⱼ and n₁ⱼ. Additionally, an α-level confidence interval for the marginal treatment effects can be estimated from the sampling distribution under the alternative hypothesis.

Embodiments of the invention can control Type I error associated with estimating treatment effects and maintain an unbiased treatment effect. As mentioned above, treatment assignment may be independent of strata in several embodiments of the invention. In some embodiments, wⱼ→_{P}P(X=j), whereby [symbol] and [symbol] may be consistent estimates of the true probabilities for all j. It follows that ψ̅→_{P}ψ̅, making a consistent estimator, and \hat{V} can also be consistent for the true sampling variance \hat{ψ} in a number of embodiments.

Process 200 estimates (260) study power based on estimated outcome distributions assuming a stratified primary analysis. In many embodiments, as N→∞, \hat{V}=V+O_{P}(n⁻¹), where V is the expected variance of the CMH estimate under some assumption about probabilities and strata weights. In certain embodiments, an assumption of wⱼ=P(X=xⱼ) may be made. In several embodiments, as sample sizes of the trials increase such that N→∞, power of the study approaches:

$${( 1 - β )}_{CMH} = ϕ ( {ϕ}^{- 1} ( \frac{α}{2} ) + \overline{\frac{ψ}{v}} ) + ϕ ( {ϕ}^{- 1} ( \frac{α}{2} ) - \overline{\frac{ψ}{v}} ) \qquad ( 1 )$$

Reduction in variances of estimation using CMH model and binary outcome predictions compared to variances of estimation that do not use binary outcome predictions may be expressed as:

$$γ = 1 - \frac{{σ}_{0 , CMH}^{2}}{{σ}_{0 , unadjusted}^{2}} \qquad ( 2 )$$

In practice, a priori approximation of equation (2) may require having expectations of some variables which can be estimated from a historical dataset.

In certain embodiments, equation (2) may be approximated by R², the squared correlation between X and Y on the control treatment Y(r_{XY}). In some embodiments, the Spearman correlation may be used to determine the association between X and Y, since X may be defined as a categorical ordinal covariate, and Y may be defined as a categorical binary outcome. In several embodiments, other meaningful measures such Kendall's tau or Area Under the Curve (AUC) may be used to determine the level of association.

In numerous embodiments, the variance of the treatment effect estimated by the CMH test, σ_{CMH}², is also a function of strata-level outcomes. When values of J and p₀ⱼ are known for all strata, E(γ) can be calculated as the expected value. When the values of design parameters are limited, another a priori process may be required to estimate strata possibilities. In several embodiments, the process requires parameters J, p̅₀, and r_{XY} to be simulated for a sample size N. Subjects in the simulated data can be assigned to strata with outcomes (xᵢ, yᵢ), where pₒⱼ can be taken as the means. Under certain assumptions, variance reduction can be approximated by:

$${R}^{2} γ = 1 - \frac{\frac{1}{J} {\sum }_{1}^{J} 𝔼 [ 𝕍 ( {x}_{j} ) ]}{𝔼 [ 𝕍 ( {\overline{p}}_{0} ) ]} \qquad ( 3 )$$

where V(xⱼ) is the expected variance for stratum xⱼ based on the estimated pₒⱼ. In practice, formal estimation of σ² for both the CMH and unadjusted tests should be performed using expected parameter values as described above.

Embodiments of the invention can reduce the control arm sample size necessary for RCTs while maintaining desired power and type I error control. Let n*₀ be the control arm sample size under the CMH test, and n₀ be the control arm sample size from an unadjusted test. In several embodiments, process approximate the reduction in sample size

$$1 - \frac{{n}_{0}^{⋆}}{{n}_{0}}$$

a prior oy solving:

[symbol]

[σ_{1,unadjusted}²]= [symbol] [\hat{V}*₁] (4)

where subscript 1 denotes the value under the alternative hypothesis given above.

While specific processes for estimating treatment effects for binary outcomes using stratification in RCTs are described above, any of a variety of processes can be utilized to estimating treatment effects for binary outcomes using stratification in RCTs as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

### Estimating TTE Treatment Effects

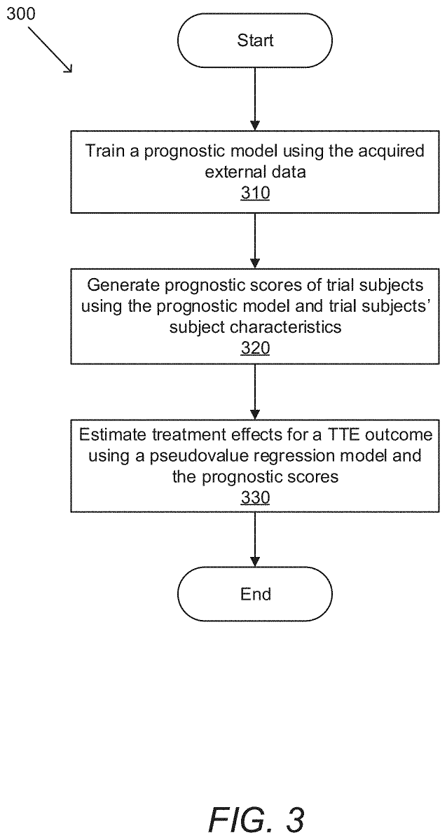

TTE endpoints refer to the time point where certain events occur in a trial. Treatment effects detected from TTE endpoints can be another indicator of efficacy of new treatments. Different trial subjects may progress differently, and detected differences in subjects' TTE between treatment and control conditions can assist researchers with making potential improvements to medicine. A conceptual illustration of the estimating TTE treatment effects using pseudovalue regression with a covariate acquired from a generative model is illustrated in FIG. 3. In many embodiments, the event of interest for purposes of estimating TTE treatment effects is whether trial subjects have a favorable or unfavorable outcome on study, accounting also for intercurrent events. Process 300 trains (310) a prognostic model using the acquired external data. In some embodiments, external data may be from control arms of clinical trials, high quality observational studies, or any other data source that can approximate high quality datasets. External data in accordance with several embodiments of the invention may include subject characteristics of trial subjects and their eventual trial outcomes from the previous randomized clinical trials. In several embodiments, the prognostic model may be a simple rules-based model. The prognostic model may be a model-based generative machine learning model in certain embodiments.

Process 300 generates (320) prognostic scores for trial subjects using the trained prognostic model and subjects' subject characteristics. In certain embodiments, prognostic scores may be expected values of treatment outcome predictions predicted by the prognostic model. Prognostic scores may be defined by cᵢ:=f(xᵢ¹, . . . , xᵢ^{N}) where Xᵢ represents the ith potentially prognostic baseline characteristic. In a number of embodiments, processes can calculate expected values of outcome predictions by drawing samples from the prognostic model and applying the Monte Carlo method on the drawn samples.

Process 300 estimates (330) treatment effects for a TTE outcome using a pseudovalue regression model and prognostic scores. In certain embodiments, processes perform this estimation after the completion of target trial where available TTE data may be readily collected. In many embodiments, the time to event of interest may be restricted mean survival times (RMST). Processes in accordance with several embodiments of the invention fits a generalized linear model (GLE) to TTE data including the censored data. Let θ=E[f(x)] for some function f where θ denotes the RMSTs, and Xᵢ, . . . , Xₙ represents independent and identically distributed quantities. Let θᵢ=E[f(Xᵢ)|z₁] be the conditional expectation of f(Xᵢ) given zᵢ, where zᵢ, . . . , zₙ represents independent and identically distributed samples of covariates. In a number of embodiments, an unbiased estimator \hat{θ} of θ may be used to define the i^{th} pseudo-observation of θ as:

\hat{θ}ᵢ=n\hat{θ}−(n−1)\hat{θ}⁻ⁱ (5)

where \hat{θ}⁻ⁱ is a jackknife leave-one-out estimator of θ based on {Xⱼ:j≠i}. In several embodiments, linear model θᵢ=β₀+β₁1_{T}+β₂cᵢ may be used to solve β=(β₀, β₁, β₂) from the following estimation equation:

$${\sum }_{i} {U}_{i} ( β ) = {\sum }_{i} \frac{\partial {θ}_{i}^{′}}{\partial β} ( \hat{{θ}_{ι}} - {θ}_{i} ) = 0 \qquad ( 6 )$$

Coefficient β₂ may be estimated, and a null hypothesis may be assessed by computing a two-sided p-value based on a t-distribution in accordance with embodiments of the invention. Pseudovalues \hat{θ}ᵢ substitute the observed data X in the model. This can serve as a work around, as it models censored data in the same way as uncensored data. Prognostic score c in covariate adjusted pseudovalue regression provides a coefficient estimation with higher precision. In many embodiments, as the correlation between covariate and pseudovalue increases, gain in precision may be greater. In some embodiments, increased precision can be used to boost efficiency and/or to reduce sample size.

In select embodiments, processes may obtain the greatest gain in variance reduction by fitting a survival model P to provide estimates of the conditional survival distribution for each trial subject i. In several embodiments, the estimates of conditional survival distribution may be represented by cᵢ:=E[p_{P}(X>t|xᵢ¹, . . . , xᵢ^{N})].

In many embodiments, processes can reduce the sample size of the trial by estimating the correlation between cᵢ and \hat{θ}ᵢ. In a number of embodiments, the estimation of correlation for trial subjects may be based on a testing data set in the external data and expected treatment effects in the target trial, where correlation may be estimated based on the similarity between the external data and the target trial. Estimated correlation may be deflated if outcomes presented in the target trial differ from external data. In some embodiments, the estimated correlation can be used for sample size calculation in the design stage of the trial. In many embodiments, process will maintain type I error and produce unbiased estimates of treatment effects.

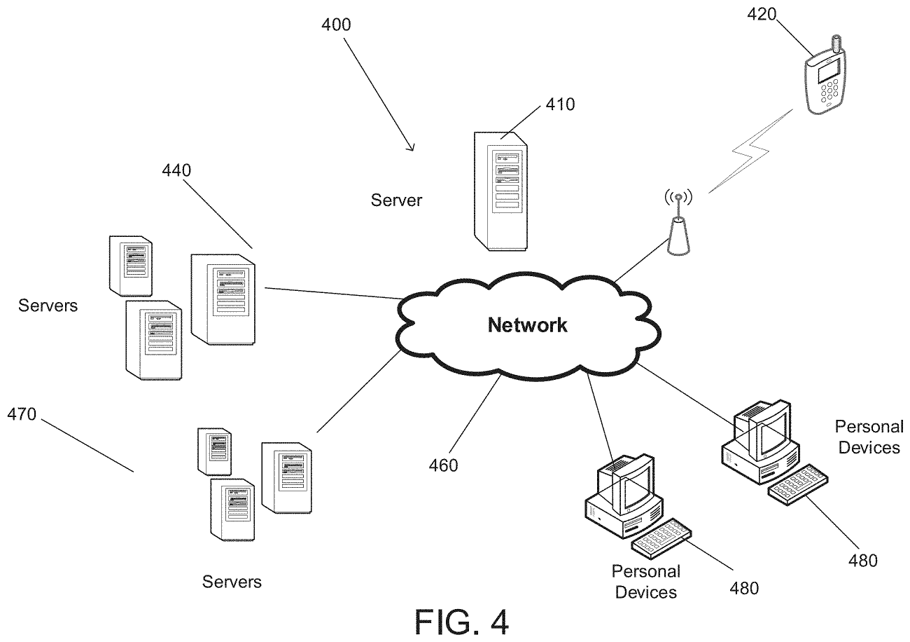

An example of a network that processes described above can be implemented on in some embodiments of the invention is illustrated in FIG. 4. In many embodiments, network 400 includes a communication network 460. Communication network 460 may be a network such as the Internet that allows devices connected to the network 460 to communicate with other connected devices. In a number of embodiments, server systems 440 and 470 can be connected to the network 460. According to various embodiments of the invention, each of the server systems 440 and 470 may be a group of one or more servers communicatively connected to one another via internal networks that execute processes that provide cloud services to users over the network 460. For purposes of this discussion, cloud services are one or more applications that are executed by one or more server systems to provide data and/or executable applications to devices over a network.

The server systems 440 and 470 are shown each having three servers in the internal network. However, the server systems 440 and 470 may include any number of servers and any additional number of server systems may be connected to the network 460 to provide cloud services. In some embodiments, there may only be a single server 410 that is connected to network 460 to provide services to users. In accordance with various embodiments of this invention, a computing system that uses systems and methods that estimate treatment effects in a randomized controlled trial in accordance with an embodiment of the invention may be provided by a process being executed on a single server system and/or a group of server systems communicating over network 460.

Users may use personal devices 480 that connect to the network 460 to perform processes that estimate treatment effects in a randomized controlled trial in accordance with various embodiments of the invention. In the shown embodiment, the personal devices 480 are shown as desktop computers that are connected via a conventional “wired” connection to the network 460. However, personal device 480 may be a desktop computer, a laptop computer, a smart television, an entertainment gaming console, or any other device that connects to the network 460 via a “wired” connection. Mobile device 420 can connect to network 460 using a wireless connection. A wireless connection may be a connection that uses Radio Frequency (RF) signals, Infrared signals, or any other form of wireless signaling to connect to the network 460. In the example of this figure, the mobile device 420 is a mobile telephone. However, mobile device 420 may be a mobile phone, Personal Digital Assistant (PDA), a tablet, a smartphone, or any other type of device that connects to network 460 via wireless connection without departing from this invention.

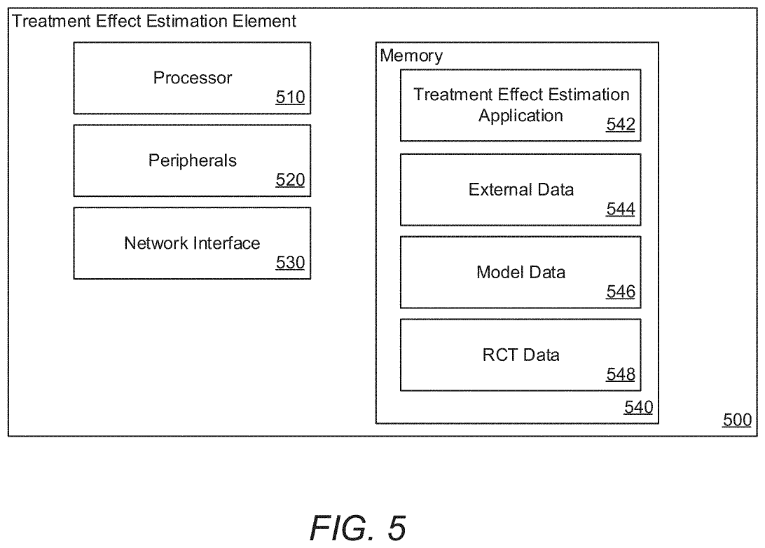

An example of a computing system that processes described above can be implemented on in some embodiments of the invention is illustrated in FIG. 5. Treatment effect estimation element 500 includes a network interface 530 that can receive external data, and a memory 530 to store the external data under an external data memory 544. Processor 510 may execute the treatment effect estimation application 542 to estimate treatment effects in a randomized controlled trial in accordance with several embodiments of the invention. One skilled in the art will recognize that the computing system may exclude certain components and/or include other components that are omitted for brevity without departing from this invention.

In many embodiments, processor 510 can include a processor, a microprocessor, controller, or a combination of processors, microprocessor, and/or controllers that performs instructions stored in the memory 540 to manipulate trial data stored in the memory. Processor instructions can configure the processor 510 to perform processes in accordance with certain embodiments of the invention. In various embodiments, processor instructions can be stored on a non-transitory machine readable medium.

Although a specific example of a treatment effect estimation element 500 is illustrated in this figure, any of a variety of treatment effects estimation elements can be utilized to perform processes for estimating treatment effects in RCTs similar to those described herein as appropriate to the requirements of specific applications in accordance with embodiments of the invention.

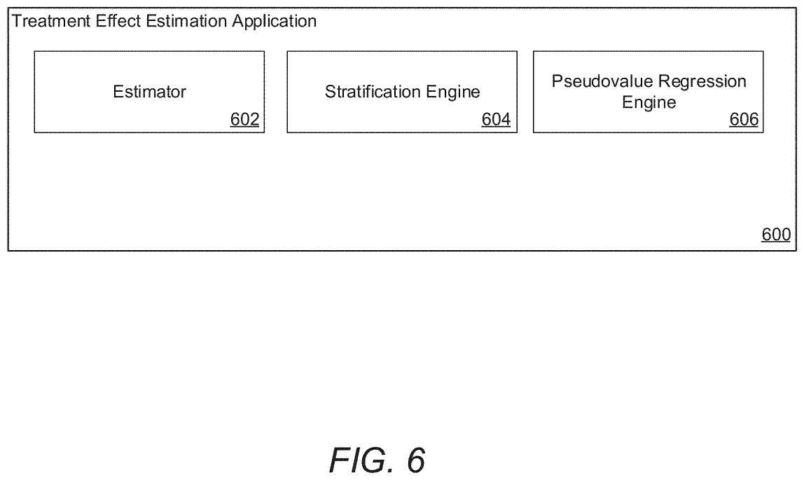

An example of an estimation application that executes instructions to estimate treatment effects in a randomized controlled trial in accordance with an embodiment of the invention is illustrated in FIG. 6. In several embodiments, estimation application 600 may include an estimator 602, a stratification engine 604, and a pseudovalue regression engine. Estimator 602 in accordance with various embodiments of the invention can be used to estimate treatment effects in a randomized controlled trial. In several embodiments, the stratification engine 604 can be used to stratify the trial subjects for estimating treatment effects for binary outcomes. In some embodiments, the pseudovalue regression engine 606 can be used to estimate TTE treatment effects of trial subjects.

Although a specific example of treatment effect estimation application is illustrated in this figure, any of a variety of treatment effect estimation applications can be utilized to perform processes for estimating treatment effects in RCTs similar to those described herein as appropriate to the requirements of specific applications in accordance with embodiments of the invention.

Although specific methods of estimating treatment effects in an RCT are discussed above, many different design methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for estimating treatment effects in randomized controlled trials, the method comprising:
receiving external data of previous randomized clinical trials;
generating sets of one or more subject characteristics of a plurality of trial subjects;
estimating binary outcomes of trial subjects using a stratification process; and
estimating time-to-event (TTE) treatment effects of trial subjects using pseudovalue regression.

2. The method of claim 1, where estimating binary outcomes of trial subjects using a stratification process comprises:
training a prognostic model using the received external data;
generating outcome predictions for trial subjects using the prognostic model;
defining a variable to stratify the trial subjects based on the outcome predictions;
stratifying all trial subjects by the variable in to a plurality of strata; and
estimating treatment outcomes for trial subjects in all strata.

3. The method of claim 1, where estimating TTE treatment effects of trial subjects using pseudovalue regression comprises:
training a prognostic model using the received external data;
generating prognostic scores of trial subjects using the prognostic model and the generated trial subjects' subject characteristics; and
estimating TTE treatment effects for trial subjects using a pseudovalue regression model and the prognostic scores.

4. The method of claim 1, where the sets of one or more characteristics of a plurality of trial subjects comprises baseline covariates of trial subjects, and treatment assignments of trial subjects.

5. The method of claim 2, where the prognostic model is a generative model.

6. The method of claim 2, where the prognostic model is a generalized linear model.

7. The method of claim 3, where the prognostic model is a simple rules-based model.

8. The method of claim 3, where the prognostic model is a model-based generative machine learning model.

9. The method of claim 3, where estimating TTE treatment effects comprises estimating restricted mean survival times of trial subjects.

10. The method of claim 1, further comprising designing clinical studies based on the estimated treatment effects.

11. A non-transitory machine readable medium containing processor instructions for estimating treatment effects in randomized controlled trials, where execution of the instructions by a processor causes the processor to perform a process that comprises:
receiving external data of previous randomized clinical trials;
generating sets of one or more subject characteristics of a plurality of trial subjects;
estimating binary treatment outcomes of trial subjects using a stratification process; and
estimating time-to-event (TTE) treatment effects of trial subjects using pseudovalue regression.

12. The non-transitory machine readable medium of claim 11, where estimating binary outcomes of trial subjects using a stratification process comprises:
training a prognostic model using the received external data;
generating outcome predictions for trial subjects using the prognostic model;
defining a variable to stratify the trial subjects based on the outcome predictions;
stratifying all trial subjects by the variable in to a plurality of strata; and
estimating treatment outcomes for trial subjects in all strata.

13. The non-transitory machine readable medium of claim 11, where estimating TTE treatment effects of trial subjects using pseudovalue regression comprises:
training a prognostic model using the received external data;
generating prognostic scores of trial subjects using the prognostic model and the generated trial subjects' subject characteristics; and
estimating TTE treatment effects for trial subjects using a pseudovalue regression model and the prognostic scores.

14. The non-transitory machine readable medium of claim 11, where the sets of one or more characteristics of a plurality of trial subjects comprises baseline covariates of trial subjects, and treatment assignments of trial subjects.

15. The non-transitory machine readable medium of claim 12, where the prognostic model is a generative model.

16. The non-transitory machine readable medium of claim 12, where the prognostic model is a generalized linear model.

17. The non-transitory machine readable medium of claim 13, where the prognostic model is a simple rules-based model.

18. The non-transitory machine readable medium of claim 13, where the prognostic model is a model based generative machine learning model.

19. The non-transitory machine readable medium of claim 13, where estimating TTE treatment effects comprises estimating restricted mean survival times of trial subjects.

20. The non-transitory machine readable medium of claim 11, further comprising designing clinical studies based on the estimated treatment effects.
